(19) United States
(12) Patent Application Publication
(10) Pub. No.: US 2020/0276395 A1
(43) Pub. Date: Sep. 3, 2020

(54) A NEEDLE DEVICE FOR SELECTIVE SUBCUTANEOUS FLUID INJECTION

(71) Applicant: PERFACTION TECHNOLOGIES LTD, Rehovot (IL)

(72) Inventors: Shy Zyman, Pardes Hana Karkur (IL); Hagai Aharon Sroussi, Machane Yatir (IL)

(21) Appl. No.: 16/650,213

(22) PCT Filed: May 8, 2018
(86) PCT No.: PCT/IL2018/050501

§ 371 Date: Mar. 24, 2020

(30) Foreign Application Priority Data
Sep. 24, 2017 (US) 62562435

(51) Int. Cl. A61M 5/32; A61M 5/46

(52) U.S. Cl. CPC A61M 5/3291; A61M 2202/08; A61M 5/46

(57) ABSTRACT

This invention is directed to a novel needle device for selective subcutaneous fluid injection, the needle device comprising at least a connector configured to allow connection of the needle device with injecting means; a spacer configured to be connected with a needle in a right angle so as to enforce vertical insertion of the needle into a body of a treated object for controlling the penetration depth of the needle into the body of the treated object; and a needle having a sealed upper segment, a perforated lower segment and a blocked bottom end, said perforated lower segment containing multiple micro holes to selective allow horizontal dispersion of fluid into a target layer within the body of said subject at the surroundings of the perforated lower segment. This invention is further directed to a method for injecting fluid to a subcutaneous target layer to be treated with the novel needle device of the invention.

### TECHNOLOGICAL FIELD

The present invention relates to selective injection of fluid into the body of a treated object. More particularly, the present invention is directed to novel needle device for selective subcutaneous fluid injection.

### BACKGROUND

In many medical and cosmetic procedures there is a need to transfer fluids to areas below the surface of skin (e.g. subcutaneously). Even though that the most common method for injecting fluids subcutaneously to a desired area is by using a needle connected to a syringe it comprises a lot of shortcomings. Usually, the use of needles causes damage to the skin that requires a long healing period. Therefore, to minimize the damage, the administrator must be trained in order to perform the procedure correctly, at the exact position to get the desired results. Furthermore, the fluid is distributed over a relatively small area that is close to the injection spot and not evenly.

The field of subcutaneous fluid injection devices has developed greatly in recent years with a lot of needle jet injectors emerging having advantages over traditional syringes injection. Concurrently, multiple-hole needles have started to emerge in the art. Various usages of multiple-hole needles and devices attached thereto are known. Some examples are provided in the following references: U.S. Pat. Nos. 8,038,664; 6,969,373; 8,083,722; and 5,709,668.

In general, U.S. Pat. No. 8,038,664 is directed to an apparatus for delivering a quantity of fluid to bone marrow of a bone or providing access to remove fluids from a target site is provided; U.S. Pat. No. 6,969,373 describes a medical device having a needle or a catheter, insertable into a living body, which defines a plurality of holes in fluid communication with a central lumen; U.S. Pat. No. 8,083,722 disclose medical delivery devices that can be used to effectively distribute a medical agent to multiple sites within a tissue volume without requiring the device to be repositioned, for example distribution of a medical agent within the nucleus pulposus tissue of a spinal disc. This invention further describes medical delivery devices for simultaneously remove fluid from the tissue volume into which a medical agent is being delivered for avoiding or decreasing any pressure that may be build-up; U.S. Pat. No. 5,709,668 is directed to an automatic medicament injector employing a non-coring needle having side port geometry optimized to minimize or eliminate the coring of a rubber seal or septum when impaled by the internal needle tip of the cannula. The geometry avoids direct exposure of the butt end face of the needle to the rubber seal by providing a crimp to the material at the butt end and openings in the side thereof using electro-discharge machining (EDM) processes providing plural openings therein for introduction of medicament or aspiration through the side port geometry.

In view of the above, there is a need in the art for simple and effective means for selectively and efficiently inject fluids to a target layer with maximal and multidirectional dispersion of the injected fluid within the target layer, and yet with minimal injection repetitions, while limiting the spread of the injected fluid to the target layer and keeping the upper and the lower layers surrounding the target layer clean and unaffected by the injected material.

### General Description

The present invention is aimed to provide a novel needle device configured to selectively inject fluids to subcutaneous target layer, while keeping the other layers clean from the injected material as will be described in detail hereinbelow.

While in some known injection techniques all the skin's layers are affected by the fluid's dispersion, which may be undesirable, the novel needle device of the present invention enables to limit the injection of fluid into a specific layer of the skin without affecting the other layers. Furthermore, the unique structure of the novel needle device provides a better accuracy in the depth of penetration of the needle in a manner that even unskilled person can use the needle device and inject the fluid into a proper depth such that the fluid will be dispersed in the target are.

Thus, in one main aspect of the invention a needle device for selective subcutaneous fluid injection is provided. The needle device comprising at least:

(i) a spacer configured to be connected with a needle in a right angle so as to enforce vertical insertion of the needle into a body of a treated subject for controlling the penetration depth of the needle into a target layer to be treated; and
(ii) a needle having: a sealed upper segment; a perforated lower segment containing multiple micro holes; and a blocked bottom end; wherein, the segments and blocked bottom end allow selective horizontal multidirectional dispersion of the injected fluid into the target layer through the multiple micro holes to allow 360 spherical dispersion of the injected fluid within the target layer.

In accordance with embodiments of the invention, the vertical insertion of the needle allows to direct the injected fluids toward the target layer positioned at the depth of the needle penetration and adjacent to the lower perforated segment of the needle, such that fluid is dispersed selectively from the micro holes of the perforated lower segment into the target layer in a 360-degree spherical dispersion, while other tissues above and below the target layer that are adjacent to the sealed upper segment and the blocked bottom end, remain clean from the injected fluid.

The fluid injected into the target layer is dispersed horizontally through the micro holes positioned at the outer surface of said needle, all-around of area “c” of the needle, in a 360-degree sphere spread.

The needle length may vary according to the depth of the target layer within the body of the treated subject. Also, the ratio between the sealed upper segment of the needle and the perforated lower segment of the needle may vary in a manner that the thicker the target layer is the perforated segment portion increases relative to the sealed segment portion, so as to allow fluid to disperse horizontally into a large portion of the target layer in a single injection.

The micro holes may have either one of similar dimensions or different dimensions and have a shape of any one of the following geometrical shapes: round holes, rectangular holes, elliptical holes, pentamer holes, square holes and hexagon holes, and may be angled at the same of various angles.

In some embodiments, the needle devise further comprising a connector that is configured to allow connection of the needle device with an injection means such as but not limited to a syringe and a jet injection system. In a specific embodiment the connector may be a nozzle.

In some further embodiments, the spacer may functionally serve as a connector and allows connection of the needle device with injecting means.

In a further aspect of the invention, a kit comprising at least two needle devices according to the description above may is provided, wherein each of said needle device has a different needle length and/or a different perforated segment length and/or a different sealed portion length relative to the other needle device/s comprised in the kit, each needle device of the kit is suitable for injecting fluid into different target layer that is positioned at a different depth within a body of a treated subject.

The needle device of the invention enables better homogenous and accurate coverage of the complete skin layer being treated thanks to the 360-degree spherical dispersion of the injected fluids.

In one additional aspect of the invention a method for injecting fluid to a subcutaneous target layer to be treated is provided. The method comprising the following steps:
a. connecting a needle device of the invention to a syringe or to a jet injector or to a jet injection system filled with a selected injection fluid;
b. inserting vertically the needle device in a manner that the perforated lower segment of the needle of said needle device is positioned within a target layer within a body of a treated subject;
c. injecting the fluid horizontally through the perforated lower segment of the needle such that the injected fluid is dispersed horizontally within the target layer in a 360-degree spherical dispersion, while keeping the areas on top of the target layer and below the target layer clean of the injected fluid; and
d. ejecting the needle device from the injected layer and repeating steps (b) to (c) at a pre-determined distance from the first insertion point of the needle until coverage of the entire target layer to be treated.

The needle device of the present invention is enabling better and homogenous and accurate coverage of complete skin layer. The target layer to be treated may be for example, a lipoma and the injected fluid in this case may be a steroid.

The vertical insertion of the needle allows to direct the injected fluids toward a target layer positioned at the depth of the needle penetration and adjacent to the lower perforated segment of the needle, such that fluid is dispersed selectively from the micro holes of the perforated lower segment into the target layer in a 360-degree spherical dispersion, while other tissues above the target layer that are adjacent to the sealed upper segment and the blocked bottom end remain clean from the injected fluid. The needle length may vary according to the depth and/or the thickness of the target layer within the body of the treated subject. In addition, the ratio between the sealed upper segment of the needle and the perforated lower segment of the needle may vary in a manner that the thicker the target layer is, the perforated segment portion increases relative to the sealed segment portion so as to allow fluids to disperse horizontally into a large portion of the target tissue in a single injection.

In the proposed method, the micro holes may have either one of similar dimensions or different dimensions and have a shape of any one of the following geometrical shapes: round holes, rectangular holes, elliptical holes, pentamer holes, square holes and hexagon holes.

In one further aspect, a method for injecting fluid to a subcutaneous target layer to be treated is provided. The method comprising the following steps:
a. connecting a needle device to a syringe or to a jet injector/jet injection system filled with a selected injection fluid; said needle device comprising:
   a spacer configured to be connected with a needle in a right angle so as to enforce vertical insertion of the needle into a body of a treated subject for controlling the penetration depth of the needle into a target layer to be treated; and
   a needle having: a sealed upper segment; a perforated lower segment containing multiple micro holes; and a blocked bottom end;
wherein, said segments and blocked bottom end allow selective horizontal multidirectional dispersion of the injected fluid into the target layer through the multiple micro holes to allow 360-degree spherical dispersion of the injected fluid into the target layer;
b. inserting vertically the needle device in a manner that the perforated lower segment of the needle of said needle device is positioned within a target layer within a body of a treated subject;
c. injecting the fluid horizontally through the perforated lower segment of the needle such that the injected fluid is dispersed horizontally within the target layer in a 360-degree spherical dispersion, while keeping the areas on top of the target layer and below the target layer clean of the injected fluid; and
d. ejecting the needle device from the injected layer and repeating steps (b) to (c) at a pre-determined distance from the first insertion point until coverage of the entire target layer to be treated.

Some examples of a target layers to be treated by the novel needle device and methods described herein may be for example, a subcutaneous fat layer. In such treatment the injection fluid may comprise a lipolytic material. Alternatively, the target layer to be treated may be a lipoma and the injection fluid in such case may be a steroid.

In some aspects of the invention, the novel needle device can be integrated with a nozzle that allows connection of the needle device to a get injector/jet injection system or any other injection device available in the market to thereby allow selective jet injection of fluid horizontally and subcutaneously wherein the dispersion is limited to the target layer. In such embodiment, the integration of the novel needle device with a jet injector/jet injection system provides an accurate, selective and high-pressure fluid injection, while providing a horizontally 360-degree spherical jet that distributes the fluid evenly and over a relatively large area compared to injection by syringe.

### BRIEF DESCRIPTION OF THE DRAWINGS

Examples illustrative of embodiments of the disclosure are described below with reference to figures attached hereto. Dimensions of components and features shown in the figures are generally chosen for convenience and clarity of presentation and are not necessarily shown to scale. Many of the figures presented are in the form of schematic illustrations and, as such, certain elements may be drawn greatly simplified or not-to-scale, for illustrative clarity. The figures are not intended to be production drawings.

The figures (FIGS.) are listed below.

FIG. 1 is a schematic isometric view of a needle device in accordance with one example of the invention;

FIG. 2 is a schematic front view illustration of the needle device of FIG. 1 inserted into a body of a treated object;

FIGS. 3A-3C are schematic partial front view illustrations of three optional examples of needle devices in accordance with the present invention, in which the needle length and the proportion of the sealed area and the perforated area are different; wherein, FIG. 3A illustrates a needle device with ratio between segments “a” and “c” optimal for usage for the Submental skin thickness; FIG. 3B illustrates a needle device with a smaller ratio between segments “a” and “c” optimal for usage for the abdomen skin thickness; and FIG. 3C describes a needle device with a larger ratio between segments “a” and “c” that is optimal for usage for the face and neck skin thickness.

FIGS. 4A-4B are schematic partial front view illustrations of two exemplary needle devices in accordance with embodiments of the invention wherein the holes have a pentamer shape (FIG. 4A) and an elliptical shape (FIG. 4B);

FIG. 5 is a schematic illustration of a needle device integrated to a nozzle for usage of the novel needle device with a jet injection system in accordance with examples of the invention;

FIGS. 6A-6B are histological photographs illustrating subcutaneous injection of lipolytic material into a pig tissue with a standard needle having a single hole at the bottom part (6A); and with the needle device of the present invention having multiple side holes on the outer surface and sealed at the bottom part of the needle (6B).

### DETAILED DESCRIPTION OF EMBODIMENTS

In the following description, various aspects of a novel needle device for selective subcutaneous injection of fluids are provided. The novel needle device is configured and operable to allow vertical insertion into the body to allow accurate and controllable injection depth, with horizontal 360-degree spherical dispersion of the injected fluid throughout multiple holes positioned along the needle in a predefined area for selectively and limited delivery of fluids to a target layer with minimal damage and dispersion into adjacent layers. In accordance with embodiments of the invention the novel needle device may be connected to a syringe for injecting the desired fluid to the target layer or may be attached to other injection devices, such as but not limited to, a jet injection device as will be described in detail with reference to the drawing below. For the purpose of explanation, specific configurations and details are set forth in order to provide a thorough understanding of the invention.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment. Furthermore, it should be understood that the disclosure can be carried out or practiced in various ways, and that the disclosure can be implemented in embodiments other than the exemplary ones described herein below. The descriptions, examples and materials presented in the description, as well as in the claims, should not be construed as limiting, but rather as illustrative.

Reference is now made to the figures.

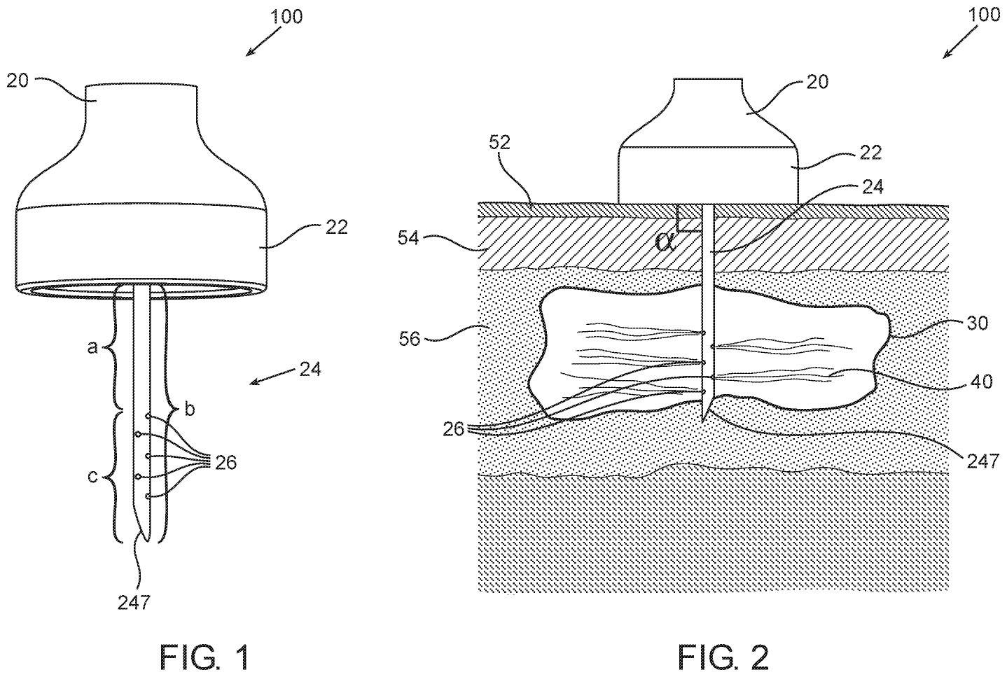

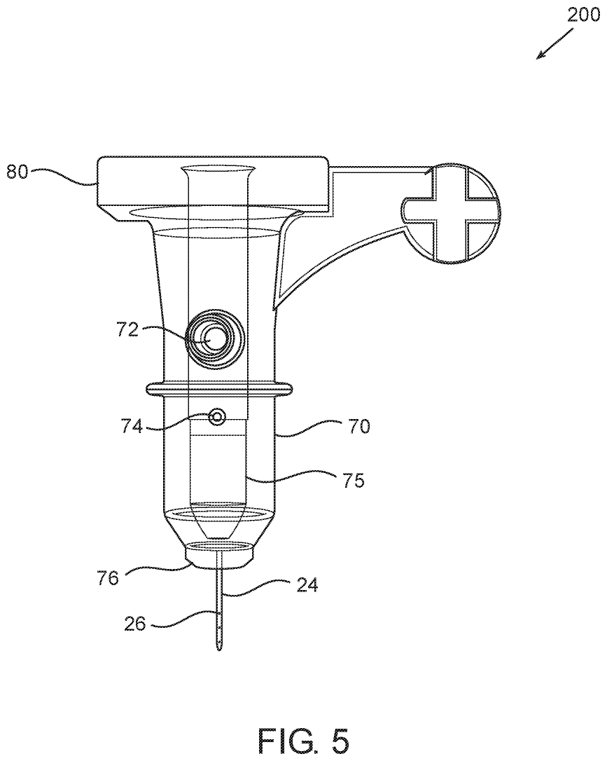

FIG. 1 is a schematic isometric view of a needle device in accordance with one optional example of the invention. In the specific example illustrated therein, needle device 100 comprised a connector 20 configured to allow connection of the needle device to injection means such as but not limited to, a syringe, an injection device, and a Louer. Connector 20 may be shaped and designed according to the tool structure that it is configured to be connected to. Therefore, the example provided herein should not be construed as limiting the scope of this invention in any manner. A specific example of a nozzle connected to a jet injection system will be described in detail with reference to FIG. 5 hereinbelow. In the example described in FIG. 1, connector 20 is connected to a spacer 22 that is physically attached to a needle 24 and configured to enforce vertical insertion of needle 24 into a target layer for injection. The term “target layer” as used herein means an area within a body of an object that the injected material is configured to be dispersed at least in part of it. The target layer may be positioned below and/or above other tissue layers that are not part of the target layer and should not be treated. The terms “target layer”, “treated layer”, “target layer” and “treated body area” are used herein interchangeably and are all directed to the same meaning.

The vertical insertion of needle 24 into the body of an object to be treated insures accuracy of penetration depth and enables controllable injection of a desired material into the target layer. This ability to control the depth of penetration and to reach a desired target layer is extremely critical when the injected material is aimed to destroy a certain tissue, such as tumor or fat layer, since any dripping of the injected material to adjacent tissues/layers may cause them undesired damage. The connection of needle 24 to spacer 22 creates a structural barrier that enforce vertical insertion of needle device 100 into the body of the treated object toward the target layer. Thus, ensuring insertion of needle 24 to a desired depth and simplify the injection process in a manner that eliminates the need of highly trained medical stuff. In addition, by forcing vertical insertion of the needle, the chances for human error and insertion of the needle device in an angle that may result in injection of the material outside the target layer decrease drastically.

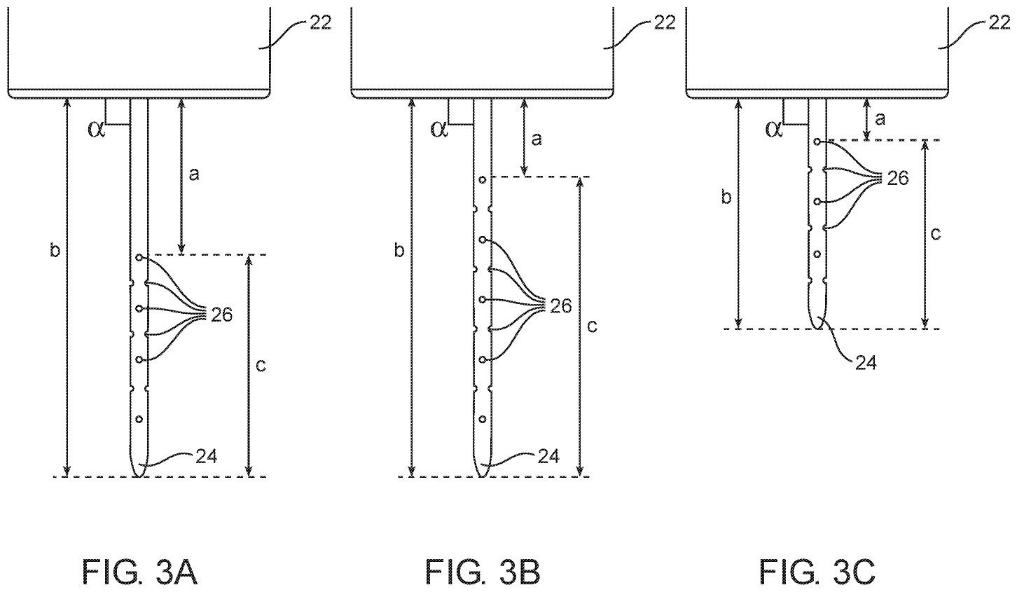

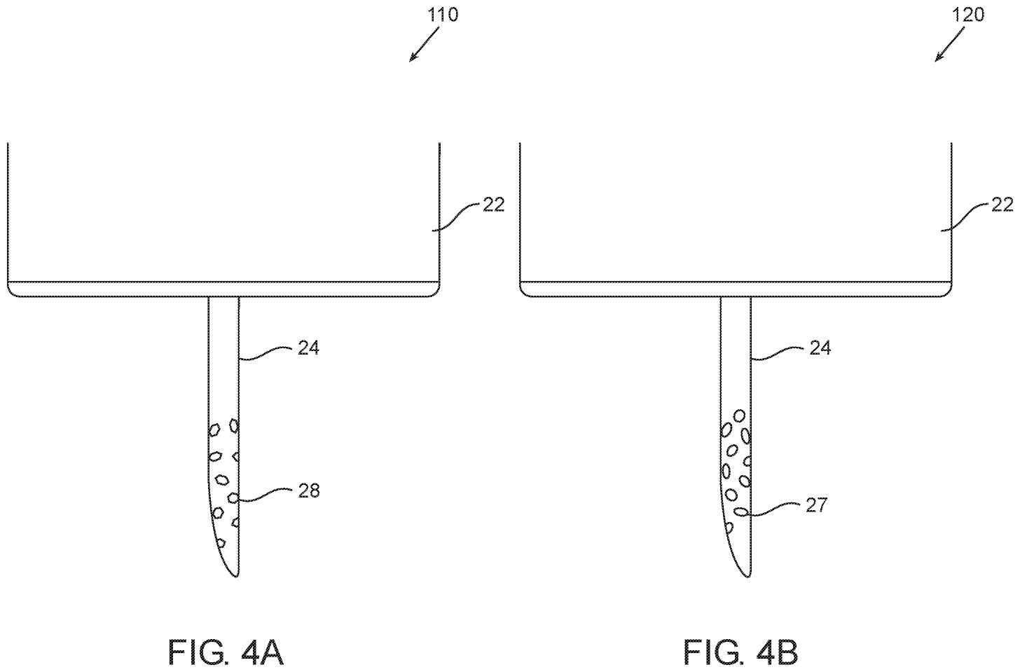

Needle 24 is preferably sealed at the bottom and comprises multiple holes 26 positioned in a predefined area on the outer surface of needle 24 along the longitudinal axis. More specifically, needle 24 allows selective horizontal delivery of fluids into the target layer, in a manner that the injected fluid is horizontally dispersed through multiple holes 26. As illustrated in this drawing, the entire needle length “b” is divided into two segments: segment “a” and segment “c”, wherein, segment “a” has a sealed outer surface that prevents exit of fluids to the surroundings of segment “a”, while segment “c” comprises multiple micro holes 26 on the outer surface of needle 24 that allows horizontal dispersion of the injected fluids into the surroundings of segment “c”. The selective horizontal dispersion of fluids via holes 26 is described in detail with reference to FIG. 2. The length “b” of needle 24 may very at least according to the depth of the target layer within the body, and the thickness of the target layer. In addition, the ratio between the sealed segment “a” and the perforated segment “c” may also vary as will be described in detail with reference to FIGS. 3A-3C. Holes 26 may be designed in various shapes such as but not limited, to round holes, elliptical holes, pentamer holes and else. The holes may be all in a similar size or in different dimensions and may be dispersed on the outer surface of needle 24 in various positions and order. Some possible examples are described with reference to FIGS. 4A-4B.

FIG. 2 is a schematic front view illustration of needle device 100 of FIG. 1 inserted into a body of a treated object and positioned within a target layer 56. As shown in the figure, connector 20 is connected to spacer 22 that forces a vertical insertion of needle 24 through the skin into the target layer due to the right angle α created between the bottom edge of spacer 22 that is parallel to the Epidermis layer 52 and perpendicular to needle 24. The vertical insertion of needle 24 allows to control the penetration depth through the skin and to ensure selective horizontal dispersion of a desired fluid 40 into target layer 56. In the specific example shown in this figure, the needle 24 is penetrating the upper layer of the skin 52 (Epidermis), the bottom layer of the skin 54 (Dermis) and the target layer 56 (fat tissue). The length of needle 24 vary from one device to the other and it is preferably designed in a manner that it ends within the target layer 56 and do not exceed beyond it, so as to prevent undesired damage that may occur during injection of fluid to other layers or tissues adjacent to the target are. Additionally, the sealed upper segment “a” ensures that fluid is dispersed only into the target layer 56 and not into adjacent layers. The holes at the perforated segments “c” and the closed tip 247 of needle 24 insures efficient horizontal dispersion of desired fluid 40 into the target layer. Thanks to the position of the holes on the needle and the horizontal, 360-degree dispersion of the fluid, the injected material is limited to the target layer 56 and it is dispersed within a large portion of the target layer relative to the dispersion of fluid achieved by injection with standard needle from its bottom tip. The treated area 30 obtained by a single insertion of the needle device 100 into the target layer and injection of fluid is also shown.

FIGS. 3A-3C are schematic partial front view illustrations of three optional examples of needle devices in accordance with the present invention, in which the needle length “b” and the proportion between the sealed segment “a” and the perforated segment “c” are different. For simplicity of explanation, connector 20 that is configured to allow the connection of the needle device of the invention to various injecting means such as syringes, injecting devices and jet injection systems is not shown. The novel needle device provided herein may be used for various applications. One none limiting usage of the novel needle device is to reduce fat layer. In such usage, different variations of the needle device may be used to optimize the results of the treatment. Some optional variations are disclosed in FIGS. 3A-3C that illustrate needles for injecting for example acids for lipolysis of fat tissue such as Kybella® (deoxycholic acid), wherein, each one of the needle devices illustrated in these figures is suitable for different target layers and further the physician may choose the most suitable needle device according to the specific personal characters of the treated person. For example, the needle device illustrated in FIG. 3A has a ratio between segments “a” and “c” optimal for usage for the brachial skin thickness; the needle device illustrated in FIG. 3B with a smaller ratio between segments “a” and “c” is optimal for usage for the abdomen skin thickness; and, the needle device with a larger ratio between segments “a” and “c” described in FIG. 3C is optimal for usage for the face and neck skin thickness.

One optional usage of the needle device of the present invention is for treating Lipoma. Some optional materials to be injected in this treatment may be Phosphatidylcholine and Deoxycholate Compound such as Sodium Deoxycholate and Deoxycholic acid. In such implementation of the invention, the needle devices illustrated in FIGS. 3A-3B may be used for treating Lipoma positioned in a deeper position within the body, wherein for a smaller size Lipoma the needle device of FIG. 3A may be used, and for a bigger size lipoma the needle device illustrated in FIG. 3B with wider “c” segment may be used to achieve maximal therapeutic effect with minimal injections. On the same concept, the needle device illustrated in FIG. 3C in which the needle length “b” is shorter compared to the needle devices illustrated in FIGS. 3A-3B may be used for treating superficial Lipoma.

In accordance with embodiments of the invention, the needle device of the invention may be arranged as a kit comprising needle devices having various lengths of needles with different ratio between the sealed segment “a” and the perforated segment “c” to enable a physician to choose the most suitable needle device for the specific treatment required and adapt it to the depth of the treatment layer, the width of the treatment layer and the type and physical properties of the injected material.

It should be clear that the examples provided above are only exemplary and other lengths and ratios between the sealed segment “a” and the perforated segment “c” are within the scope of this invention, and may vary according to the depth of the target layer (e.g. epidermis, dermis and subcutis thickness), the injected material, the state of health of the treated subject, and the treated layer (fat tissue, tumor, cellulite, etc.)

In some other embodiment of the invention the target layer is first being measured, for example by ultrasound imaging, and the appropriate needle is than selected according to the measurement for the specific treatment for obtaining optimal results.

The dimensions of the multiple-hole needle 24 may be for example in a length between 4-8 mm, and diameter between 0.2-0.6 mm. however, it should be clear that these dimensions are only none limiting exemplary sizes and the needle length and diameter can have other dimensions as well.

FIGS. 4A-4B are schematic partial front view illustrations of two exemplary needle devices 110 and 120 respectively, in accordance with embodiments of the invention wherein the holes have different shapes in each of the needle devices. In both drawings, connector 20 was removed for clarity of explanation.

FIG. 4A illustrated a needle device 110 in which the micro holes 28 have a pentamer shape. The holes are distributed on the outer surface of needle 24 in random positions i.e. horizontally, vertically or diagonally referred to the longitudinal axis of needle 24. In a similar manner, FIG. 4B illustrates a needle device 120 comprising multiple micro holes 27 having an elliptical shape. It should be clear that the shape, distribution, position, and dimensions of the micro holes may vary according to the material that is being injected and its physical characteristic such as viscosity of the injected fluid, and according to the dimensions, shape and size of the target layer. In accordance with embodiments of the invention, the micro holes may be designed in one dimension or in variety of dimensions so as to obtain different distribution patterns according to the desired therapeutic effect. In some embodiments of the invention, the micro holes may have a diameter of about 0.1-0.3 mm. In some embodiments, the micro holes may be spread uniformly around the perimeter of needle 24. Such embodiments ensure high pressure horizontal jet injection through the micro holes of needle 24 in a 360-degree spherical spread within the target layer to insurer maximal spread of the injected fluid in the target layer.

FIG. 5 illustrates one example of a needle device 200 integrated to a nozzle that is suitable for usage with a jet injection system in accordance with variations of the invention. In the specific example illustrated therein, a connector 80 is used to connect nozzle 70 to a jet injection system (not shown). The nozzle 70 may be attached to the jet injection system by pushing connector 80 into a slot on the injection system using side walls to place the nozzle 70 concentric. Also shown in this figure is connector 76 that is configured to connect nozzles 70 to multiple holes needle 24. Preferably but not necessarily, Connector 76 is integrated with multiple holes needle 24 by, for example, an injection over mold process, to establish a stable and strong integration between the multiple holes needle 24 and nozzle 70. Nozzle 70 may be designed to receive inside its inner chamber 75 a dose of fluid to be injected into the skin from a fluid entrance 72. Fluid entrance 72 may be connected to a tube that is a part of a jet injection system (not shown). In some embodiments, nozzle 70 is disposable similarly to a plastic syringe and need to be replaced between patients for sterility. In some further embodiments, nozzle 70 includes a ventilation hole 74, configured to relieve air pressure in the nozzle when fluid enters and then injected. Nozzle 70 may receive a dose of fluid, for example from a connected syringe that may be used for several subsequent injections. Alternatively, after every injection nozzle 70 is refilled precisely from the syringe for the subsequent injection, for example by using a jet injection device or any other suitable dosing system. In a preferred embodiment, multiple holes needle 24 is integrated to nozzle 70 during the production process vertically, in a manner that the needle's head is positioned perpendicular to the nozzle's longitudinal cross section axis. It should be clear that the above example illustrates one optional implementation of the invention and that the needle devise may be used with regular syringe as well.

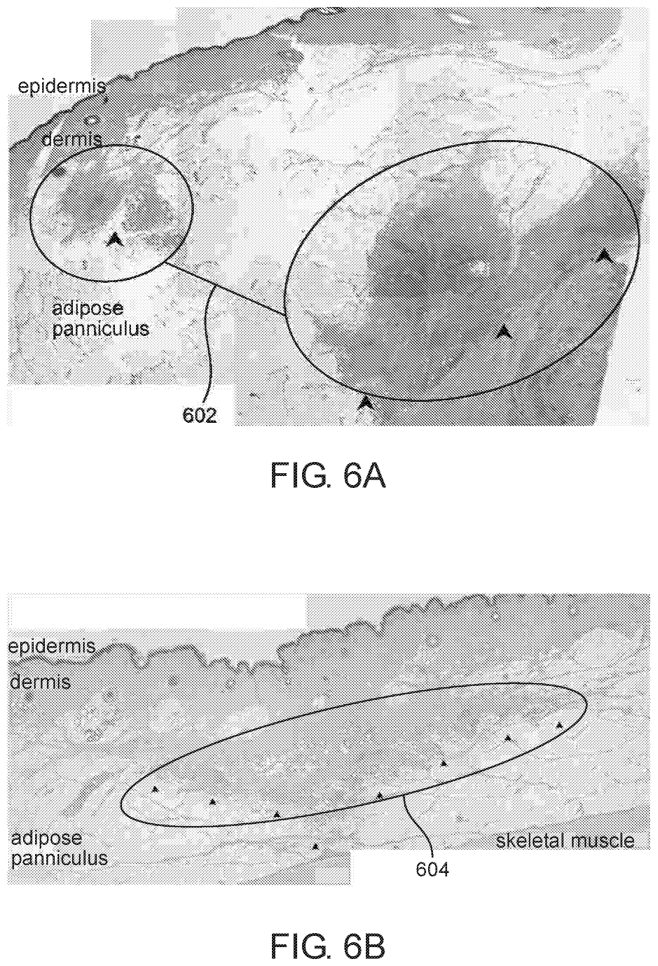

FIGS. 6A-6B are histological photographs illustrating subcutaneous injection of lipolytic material into swine skin with a regular needle having a single hole at the bottom part (6A) and with the needle device of the present invention having multiple side holes on the outer surface and sealed at the bottom part (6B). In more details, the needle that was used in this experiment has the following parameters: outside diameter: 0.33 mm; total length (b): 5.5 mm; perforated area length (c): 3.7 mm; and micro holes diameter: 0.1 mm. The arrowheads on FIG. 6A shows two foci of granuloma formation circulated by line 602 that indicate inflammatory reaction. The two foci of granuloma formation are indicating damage occurred to the dermis at the penetration area of the regular needle and further damage occurred to the underlying skeletal muscle as a result of leakage of the injected material thereto. With this regular state of the art needle the spread is bulky, inaccurate and may damage undesired layers. The larger granuloma in the adipose panniculus is 7.8 mm wide and 3.2 mm deep. On FIG. 6B the arrowheads circulated by line 604 indicate the inflammatory reaction in the superficial adipose panniculus. The lesion is 10 mm wide and 2 mm deep. The lipolysis reaction after fenestrated needle was as strong, wide and less focal with homogenous spread within the target layer. In addition, a single injection affected a wider area in the target layer, without causing damage to upper and lower adjacent layers. Also, the depth of lipolysis effect seems to be controlled by pressure of the injection. In view of the above, the affect obtained by injecting a material into a target layer with the needle device of the invention is advantageous over injection of the same material by regular needle.

It should be clear that the specific example illustrated herein is only one example and should not be construed as limiting the scope of the invention in any manner, and other connection variations of the novel needle device of the invention to a jet injector/jet injection system and/or syringe and/or other injection means should also be construed as part of this invention.

1. A needle device for selective subcutaneous fluid injection, said needle device comprising at least:
(i) a spacer configured to be connected with a needle in a right angle so as to enforce vertical insertion of the needle into a body of a treated subject for controlling the penetration depth of the needle into a target layer to be treated; and
(ii) a needle having:
a sealed upper segment;
a perforated lower segment containing multiple micro holes; and a blocked bottom end,
wherein, said segments and blocked bottom end allow selective horizontal multidirectional dispersion of the injected fluid into the target layer through the multiple micro holes to allow 360 spherical dispersion of the injected fluid within the target layer.

2. The needle device according to claim 1, wherein the vertical insertion of the needle allows to direct the injected fluids toward the target layer positioned at the depth of the needle penetration and adjacent to the lower perforated segment of the needle such that fluid is dispersed selectively from the micro holes of the perforated lower segment into the target layer in a 360-degree spherical dispersion, while other tissues above and below the target layer that are adjacent to the sealed upper segment and the blocked bottom end, remain clean from the injected fluid.

3. The needle device according to claim 1, wherein the fluid injected into the target layer is dispersed horizontally through said micro holes positioned at the outer surface of said needle.

4. The needle device according to claim 1, wherein the needle length varies according to the depth of the target layer within the body of the treated subject.

5. The needle device according to claim 1, wherein the ratio between the sealed upper segment of the needle and the perforated lower segment of the needle vary in a manner that the thicker the target layer is the perforated segment portion increases relative to the sealed segment portion, so as to allow fluid to disperse horizontally within a large portion of the target layer in a single injection.

6. The needle device according to claim 1, wherein said micro holes have either one of similar dimensions or different dimensions and have a shape of any one of the following geometrical shapes: round holes, rectangular holes, elliptical holes, pentamer holes, square holes and hexagon holes.

7. The needle device according to claim 1, further comprising a connector configured to allow connection of the needle device with an injecting means.

8. The needle device according to claim 7, wherein said injecting means are at least one of a syringe and a jet injection system.

9. The needle device according to claim 7, wherein said connector is a nozzle.

10. The needle device according to claim 1, wherein the spacer functionally serves as a connector and allows connection of the needle device with injecting means.

11. (canceled)

12. A method for injecting fluid to a subcutaneous target layer to be treated, said method comprising the following steps:
a. connecting a needle device according to claim 1 to a syringe or to a jet injector or to a jet injection system filled with a selected injection fluid;
b. inserting vertically the needle device in a manner that the perforated lower segment of the needle of said needle device is positioned within a target layer within a body of a treated subject;
c. injecting the fluid horizontally through the perforated lower segment of the needle such that the injected fluid is dispersed horizontally within the target layer in a 360-degree spherical dispersion, while keeping the areas on top of the target layer and below the target layer clean of the injected fluid; and
d. ejecting the needle device from the injected layer and repeating steps (b) to (c) at a pre-determined distance from the first insertion point of the needle until coverage of the entire target layer to be treated.

13. The method according to claim 12, wherein said target layer to be treated is a fat layer and the injection fluid comprises a lipolytic material.

14. The method according to claim 12, wherein said target layer to be treated is a lipoma and the injection fluid is a steroid.

15. The method according to claim 12, wherein the vertical insertion of the needle allows to direct the injected fluid toward a target layer positioned at the depth of the needle penetration and adjacent to the lower perforated segment of the needle, such that fluid is dispersed selectively from the micro holes of the perforated lower segment into the target layer in a 360-degree spherical dispersion, while other tissues above and below the target layer that are adjacent to the sealed upper segment and the blocked bottom end remain clean from the injected fluid.

16. The method according to claim 12, wherein the needle length varies according to the depth and/or the thickness of the target layer within the body of the treated subject.

17. The method according to claim 12, wherein the ratio between the sealed upper segment of the needle and the perforated lower segment of the needle vary in a manner that the thicker the target layer is, the perforated segment portion increases relative to the sealed segment portion so as to allow fluids to disperse horizontally into a large portion of the target tissue in a single injection.

18. The method according to claim 12, wherein said micro holes have either one of similar dimensions or different dimensions and have a shape of any one of the following geometrical shapes: round holes, rectangular holes, elliptical holes, pentamer holes, square holes and hexagon holes.

19. A method for injecting fluid to a subcutaneous target layer to be treated, said method comprising the following steps:
a. connecting a needle device to a syringe or to a jet injector/jet injection system filled with a selected injection fluid; said needle device comprising:
a spacer configured to be connected with a needle in a right angle so as to enforce vertical insertion of the needle into a body of a treated subject for controlling the penetration depth of the needle into a target layer to be treated; and
a needle having: a sealed upper segment; a perforated lower segment containing multiple micro holes; and a blocked bottom end; wherein, said segments and blocked bottom end allow selective horizontal multidirectional dispersion of the injected fluid into the target layer through the multiple micro holes to allow 360-degree spherical dispersion of the injected fluid within the target layer;
b. inserting vertically the needle device in a manner that the perforated lower segment of the needle of said needle device is positioned within a target layer within a body of a treated subject;
c. injecting the fluid horizontally through the perforated lower segment of the needle such that the injected fluid is dispersed horizontally within the target layer in a 360-degree spherical dispersion, while keeping the areas on top of the target layer and below the target layer clean of the injected fluid; and
ejecting the needle device from the injected layer and repeating steps (b) to (c) at a pre-determined distance from the first insertion point until coverage of the entire target layer to be treated.
